(19) United States
(12) Patent Application Publication
(10) Pub. No.: US 2026/0000652 A1
(43) Pub. Date: Jan. 1, 2026

(54) IMPEDING PLATINUM-BASED CHEMOTHERAPEUTIC INDUCED OTOTOXICITY USING A COLONY STIMULATING FACTOR 1 RECEPTOR INHIBITOR

(71) Applicants: THE UNITED STATES OF AMERICA, AS REPRESENTED BY THE SECRETARY, DEPARTMENT OF HEALTH & HUMAN SERVICES, Bethesda, MD (US); Washington University, St. Louis, MO (US)

(72) Inventors: Lisa Lynn CUNNINGHAM, Bethesda, MD (US); Cathy Yea Won SUNG, Bethesda, MD (US); Mark E. WARCHOL, Clayton, MO (US)

(21) Appl. No.: 18/881,825

(22) PCT Filed: Jul. 8, 2023
(86) PCT No.: PCT/US2023/069841

§ 371 Date: Jan. 7, 2025

Related U.S. Application Data
(60) Provisional application No. 63/359,593, filed on Jul. 8, 2022.

(51) Int. Cl. A61K 31/437; A61K 31/4439; A61K 31/506; A61K 31/513; A61K 33/243; A61P 27/16; A61P 35/00

(52) U.S. Cl. CPC A61K 31/437; A61K 31/4439; A61K 31/506; A61K 31/513; A61K 33/243; A61P 27/16; A61P 35/00; G01N 2500/02

(57) ABSTRACT

Disclosed is a method of impeding platinum-based chemotherapeutic induced ototoxicity, and/or other toxicity, the method comprising administering a colony stimulating factor 1 receptor (CSF1R) inhibitor to a subject in an amount sufficient to impede ototoxicity, and/or other toxicity, inducible by a platinum-based chemotherapeutic; and administering the platinum-based chemotherapeutic to the subject. The CSF1R inhibitor can be, for example, pexidartinib. The platinum-based chemotherapeutic can be, for example, cisplatin. Compositions, medicaments, kits, and uses are disclosed related to the same. Further, a method of screening for a compound able to impede a platinum-based chemotherapeutic induced toxicity is disclosed.

### CROSS-REFERENCE TO RELATED APPLICATIONS

This is an International Application under the Patent Cooperation Treaty, claiming priority to U.S. Provisional Patent Application No. 63/359,593, filed 8 Jul. 2022, the contents of which are incorporated herein by reference in its entirety.

### BACKGROUND

### Field

The present disclosure relates to methods of preventing hearing loss, ototoxicity, and toxicities generally that can be induced by platinum-based chemotherapeutics, as well as related compositions and methods of screening for compounds able to impede or prevent such toxicities.

### Description of Related Art

Cisplatin is a widely used anti-cancer drug that is toxic to mechanosensory hair cells in the inner ear and can result in significant and permanent hearing loss in pediatric and adult cancer patients. Such effects are examples of ototoxicity. Ototoxicity can be more severe in children, especially those less than 5 years old. Ototoxicity can comprise, for example, vestibular toxicity, tinnitus, hearing loss in the high frequency range (4,000 to 8,000 Hz) and/or decreased ability to hear normal conversational tones. Ototoxicity can occur during or after treatment with cisplatin Ototoxicity can be unilateral or bilateral. Deafness can occur after an initial dose of cisplatin. Sensory hair cells (HCs) are mechanoreceptors within the inner ear that play a role in the sense of hearing. HCs are formed before birth, and mammals cannot regenerate them. The cochlea converts sound to neural impulses via the organ of Corti, which is comprised of one row of inner hair cells (IHCs) and three rows of outer hair cells (OHCs). These two types of sensory HCs are separated by distinct supporting cells along the length of the cochlear duct.

Accordingly, there is a need for methods and compositions for reducing, preventing, or ameliorating such toxicities.

### BRIEF SUMMARY

The present disclosure includes the following aspects/embodiments/features in any order and/or in any combination: of any preceding or following embodiment/feature/aspect.

Disclosed is a method of impeding platinum-based chemotherapeutic induced ototoxicity, and/or other toxicity, the method comprising administering a colony stimulating factor 1 receptor (CSF1R) inhibitor to a subject in an amount sufficient to impede ototoxicity, and/or other toxicity, inducible by a platinum-based chemotherapeutic; and administering the platinum-based chemotherapeutic to the subject.

The method of any preceding or following embodiment/feature/aspect, wherein the CSF1R inhibitor comprises pexidartinib, a prodrug thereof, or a salt thereof, or any combination thereof; the platinum-based chemotherapeutic comprises cisplatin; and the ototoxicity comprises hearing loss.

The method of any preceding or following embodiment/feature/aspect, further comprising diagnosing the subject with a cancer.

The method of any preceding or following embodiment/feature/aspect, wherein the subject has been diagnosed with a cancer.

The method of any preceding or following embodiment/feature/aspect, wherein the cancer comprises a testicular cancer, a bladder cancer, a lung cancer, a stomach cancer, a head & neck cancer, or an ovarian cancer, or any combination thereof.

The method of any preceding or following embodiment/feature/aspect, wherein the cancer comprises a testicular cancer, an ovarian cancer, or a bladder cancer, or any combination thereof.

The method of any preceding or following embodiment/feature/aspect, wherein the cancer comprises a carcinoma, a sarcoma, a myeloma, a leukemia, or a lymphoma, or any combination thereof.

The method of any preceding or following embodiment/feature/aspect, wherein the cancer comprises a solid tumor.

The method of any preceding or following embodiment/feature/aspect, wherein the cancer comprises a liquid tumor.

The method of any preceding or following embodiment/feature/aspect, wherein the cancer is metastatic.

The method of any preceding or following embodiment/feature/aspect, wherein the cancer excludes a tenosynovial giant cell tumor (TGCT).

The method of any preceding or following embodiment/feature/aspect, wherein the CSF1R inhibitor comprises a small molecule therapeutic, a biologic, a prodrug thereof, or a salt thereof, or any combination thereof.

The method of any preceding or following embodiment/feature/aspect, wherein the CSF1R inhibitor comprises a small molecule therapeutic.

The method of any preceding or following embodiment/feature/aspect, wherein the CSF1R inhibitor comprises a tyrosine kinase inhibitor.

The method of any preceding or following embodiment/feature/aspect, wherein the CSF1R inhibitor comprises pexidartinib, edicotinib, PLX647, sotuletinib, vimseltinib, or imatinib, a prodrug thereof, or a salt thereof, or any combination thereof.

The method of any preceding or following embodiment/feature/aspect, wherein the CSF1R inhibitor comprises pexidartinib, a prodrug thereof, or a salt thereof, or any combination thereof.

The method of any preceding or following embodiment/feature/aspect, wherein the CSF1R inhibitor is pexidartinib, or a prodrug thereof, or a salt thereof, or any combination thereof.

The method of any preceding or following embodiment/feature/aspect, wherein the salt is a pharmaceutically acceptable salt.

The method of any preceding or following embodiment/feature/aspect, wherein the salt comprises a hydrochloride salt.

The method of any preceding or following embodiment/feature/aspect, wherein the salt comprises a monohydrochloride salt, or a dihydrochloride salt, or both.

The method of any preceding or following embodiment/feature/aspect, wherein the salt is a monohydroxychloride salt.

The method of any preceding or following embodiment/feature/aspect, wherein the CSF1R inhibitor comprises a biologic.

The method of any preceding or following embodiment/feature/aspect, wherein the CSF1R inhibitor comprises an antibody or antigen-binding fragment thereof.

The method of any preceding or following embodiment/feature/aspect, wherein the CSF1R inhibitor comprises a monoclonal antibody.

The method of any preceding or following embodiment/feature/aspect, wherein the CSF1R inhibitor comprises a chimeric antibody.

The method of any preceding or following embodiment/feature/aspect, wherein the CSF1R inhibitor comprises a humanized antibody.

The method of any preceding or following embodiment/feature/aspect, wherein the CSF1R inhibitor comprises cabiralizumab, LY3022855, emactuzumab, axatilimab, AMG820, a prodrug thereof, or a salt thereof, or any combination thereof.

The method of any preceding or following embodiment/feature/aspect, wherein the CSF1R inhibitor inhibits proto-oncogene receptor kinase (c-KIT), or FMS-like tyrosine kinase 3 with internal tandem duplication mutation (FLT3-ITD), or both.

The method of any preceding or following embodiment/feature/aspect, wherein the CSF1R inhibitor comprises an inhibitor selective to CSF1R relative to proto-oncogene receptor kinase (c-KIT), or FMS-like tyrosine kinase 3 with internal tandem duplication mutation (FLT3-ITD), or both.

The method of any preceding or following embodiment/feature/aspect, wherein the CSF1R inhibitor comprises a reversible inhibitor, or an irreversible inhibitor, or both.

The method of any preceding or following embodiment/feature/aspect, wherein the CSF1R inhibitor comprises a competitive inhibitor, or a non-competitive inhibitor, or both.

The method of any preceding or following embodiment/feature/aspect, wherein the CSF1R inhibitor antagonizes binding to CSF1R, or signaling through CSF1R, or both of CSF1, or IL-34, or both.

The method of any preceding or following embodiment/feature/aspect, wherein the platinum-based chemotherapeutic comprises a chemotherapeutic capable of forming a covalent adduct with DNA of the subject.

The method of any preceding or following embodiment/feature/aspect, wherein the platinum-based chemotherapeutic comprises cisplatin, carboplatin, oxaliplatin, paraplatin, nedaplatin, triplatin tetranitrate, phenanthriplatin, picoplatin, satraplatin, a prodrug thereof, or a salt thereof, or any combination thereof.

The method of any preceding or following embodiment/feature/aspect, wherein the platinum-based chemotherapeutic comprises cisplatin, carboplatin, oxaliplatin, or a prodrug thereof, or a salt thereof, or any combination thereof.

The method of any preceding or following embodiment/feature/aspect, wherein the platinum-based chemotherapeutic comprises cisplatin.

The method of any preceding or following embodiment/feature/aspect, wherein the platinum-based chemotherapeutic is cisplatin.

The method of any preceding or following embodiment/feature/aspect, wherein the ototoxicity comprises hearing loss, loss of balance, or tinnitus, or any combination thereof.

The method of any preceding or following embodiment/feature/aspect, wherein the ototoxicity comprises hearing loss.

The method of any preceding or following embodiment/feature/aspect, wherein the hearing loss comprises sensorineural hearing loss, bilateral hearing loss, progressive hearing loss, irreversible hearing loss, or high frequency hearing loss, or any combination thereof.

The method of any preceding or following embodiment/feature/aspect, wherein the ototoxicity comprises mechanosensory inner ear hair cell toxicity.

The method of any preceding or following embodiment/feature/aspect, wherein the mechanosensory inner ear hair cell comprises an inner hair cell, or an outer hair cell, or both.

The method of any preceding or following embodiment/feature/aspect, wherein the mechanosensory inner ear hair cell comprises an outer hair cell.

The method of any preceding or following embodiment/feature/aspect, wherein the hearing loss is associated with a threshold shift in auditory brainstem response (ABR).

The method of any preceding or following embodiment/feature/aspect, wherein the hearing loss is associated with a decrease in amplitude of distortion product otoacoustic emissions (DPOAE).

The method of any preceding or following embodiment/feature/aspect, wherein the CSF1R inhibitor inhibits macrophages, or microglia, or both.

The method of any preceding or following embodiment/feature/aspect, wherein the CSF1R inhibitor inhibits macrophages.

The method of any preceding or following embodiment/feature/aspect, wherein the macrophages comprise cochlear resident macrophages, or recruited macrophages, or both.

The method of any preceding or following embodiment/feature/aspect, wherein the resident macrophages comprise perivascular resident macrophage-like melanocytes (PVM/Ms).

The method of any preceding or following embodiment/feature/aspect, wherein the CSF1R inhibitor affects the blood labyrinth barrier (BLB).

The method of any preceding or following embodiment/feature/aspect, further comprising testing the hearing of the subject.

The method of any preceding or following embodiment/feature/aspect, wherein testing the hearing of the subject is performed before administering the platinum-based chemotherapeutic, or after administering the platinum-based chemotherapeutic, or both.

The method of any preceding or following embodiment/feature/aspect, wherein testing the hearing of the subject comprises testing for threshold shifts in auditory brainstem responses (ABR), or amplitude of distortion product-otoacoustic emissions (DPOAE), or both.

The method of any preceding or following embodiment/feature/aspect, wherein the amount of CSF1R inhibitor administered is sufficient to impede weight loss inducible by the platinum-based chemotherapeutic, impede inner ear hair loss inducible by the platinum-based chemotherapeutic, impede outer hair dysfunction inducible by the platinum-based chemotherapeutic, impede entry of cisplatin into the inner ear, impede hematological toxicity induced by the platinum-based chemotherapeutic, or any combination thereof.

The method of any preceding or following embodiment/feature/aspect, wherein the hematological toxicity comprises myelosuppression.

The method of any preceding or following embodiment/feature/aspect, wherein the CSF1R inhibitor is administered before, during, or after, or any combination thereof, administration of the platinum-based chemotherapeutic.

The method of any preceding or following embodiment/feature/aspect, wherein the CSF1R inhibitor is administered before the platinum-based chemotherapeutic.

The method of any preceding or following embodiment/feature/aspect, wherein the platinum-based chemotherapeutic is administered from one time to ten times subsequent to administration of the CSF1R inhibitor.

The method of any preceding or following embodiment/feature/aspect, wherein the CSF1R inhibitor is administered before one to ten administrations of the platinum-based chemotherapeutic.

The method of any preceding or following embodiment/feature/aspect, wherein the platinum-based chemotherapeutic is administered at least three times subsequent to administration of the CSF1R inhibitor.

The method of any preceding or following embodiment/feature/aspect, wherein the CSF1R inhibitor is administered at from about 0.5 mg to about 500 mg per dose.

The method of any preceding or following embodiment/feature/aspect, wherein the CSF1R inhibitor is administered in solid dosage form.

The method of any preceding or following embodiment/feature/aspect, wherein the platinum-based chemotherapeutic is administered at from about 0.5 mg to about 500 mg per dose.

The method of any preceding or following embodiment/feature/aspect, wherein the platinum-based chemotherapeutic is administered intravenously.

The method of any preceding or following embodiment/feature/aspect, wherein the CSF1R inhibitor and platinum-based chemotherapeutic are administered in admixture.

The method of any preceding or following embodiment/feature/aspect, wherein the subject is a mammal.

The method of any preceding or following embodiment/feature/aspect, wherein the subject is a rodent or a primate.

The method of any preceding or following embodiment/feature/aspect, wherein the subject is a companion animal.

The method of any preceding or following embodiment/feature/aspect, wherein the subject is a human.

The method of any preceding or following embodiment/feature/aspect, wherein the subject is a juvenile.

The method of any preceding or following embodiment/feature/aspect, wherein the subject is an adult.

The method of any preceding or following embodiment/feature/aspect, further comprising administering radiation to the subject, or administering at least one non-platinum-based chemotherapeutic to the subject, or both.

A pharmaceutical composition is provided comprising the CSF1R inhibitor, the platinum-based therapeutic, and a pharmaceutically acceptable excipient.

A kit is provided comprising the CSF1R inhibitor and the platinum-based chemotherapeutic.

A method of screening for a compound able to impede a platinum-based chemotherapeutic induced toxicity is provided, the method comprising the following steps. A physiologic parameter is measured in a non-human subject to obtain a first measurement, the physiological parameter associated with the toxicity and deleteriously affected by a platinum-based chemotherapeutic. A test compound is administered to the non-human subject after measuring the physiologic parameter. The platinum-based chemotherapeutic is administered to the non-human subject after measuring the physiologic parameter. The physiologic parameter in the non-human subject subsequent to administration of the test compound and the platinum-based chemotherapeutic is measured to obtain a second measurement. The first and second measurements are compared, a smaller deleterious change, than that experienced by a non-human subject not administered the test compound, indicating the test compound as a therapeutic capable of impeding the toxicity.

The method of any preceding or following embodiment/feature/aspect, wherein the toxicity is an ototoxicity.

A therapeutic identified by the method of any preceding or following embodiment/feature/aspects is provided.

A colony stimulating factor 1 receptor (CSF1R) inhibitor is provided for use in impeding ototoxicity inducible by a platinum-based chemotherapeutic.

Use of a colony stimulating factor 1 receptor (CSF1R) inhibitor is provided for impeding ototoxicity inducible by a platinum-based chemotherapeutic.

Use of a colony stimulating factor 1 receptor (CSF1R) inhibitor is provided for manufacture of a medicament to impede ototoxicity inducible by a platinum-based chemotherapeutic.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1, panel A, shows a schematic depiction of a clinically relevant mouse model in accordance with the present disclosure. FIG. 1, panel B, shows a graph of auditory brainstem response (ABR) in accordance with the present disclosure. FIG. 1, panel C, shows a graph of Distortion OtoAcoustic Emissions (DPOAE) in accordance with the present disclosure. FIG. 1, panel D, shows staining using Myosin 7a turquoise dye of outer hair cells (OHC, top) and inner hair cells (IHC, bottom) in accordance with the present disclosure.

FIG. 2, panel A, shows an image of the cochlea with DAPI and CXCR1-GFP staining under the experimental conditions (pexidartinib), demonstrating ablation of macrophages. FIG. 2, panel B, shows an image of the cochlea with DAPI and CXCR1-GFP staining under control conditions (no pexidartinib), demonstrating abundance of macrophages compared to the experimental image.

FIG. 3, panels A and C show an image of the cochlea with DAPI and CXCR1-GFP staining under control conditions (no pexidartinib), demonstrating abundance of macrophages compared to the experimental image. FIG. 3, panels B and C, show an image of the cochlea with DAPI and CXCR1-GFP staining under the experimental conditions (pexidartinib), demonstrating ablation of macrophages.

FIG. 4 shows a schematic depiction of a clinically relevant mouse model designed to examine if macrophages modulate hair cell survival or death in response to cisplatin treatment in vivo in accordance with the present disclosure.

FIG. 5 shows a graph of significantly reduced cisplatin-induced weight loss with macrophage ablation by PL3397 (pexidartinib) treatment in accordance with the present disclosure.

FIG. 6, panels A-D show enlargements of the image shown in panel E, of a cochlear section with DAPI and CX3CR1-GFP staining demonstrating that PL3397 (pexidartinib) treatment effectively ablates cochlear macrophages of both saline and cisplatin-treated mice; panel F shows a bar graph demonstrating the same in accordance with the present disclosure. For reference, panel G shows an illustration of a cross-section of the cochlear highlighting structures relevant to the blood labyrinthine barrier (BLB).

FIG. 7 shows a graph of ABR data for macrophage ablation by PLX3397 (pexidartinib) significantly reducing cisplatin-induced hearing loss in accordance with the present disclosure.

FIG. 8, panes A-D, show graphs of DPOAE data for three controls and experimental (cisplatin plus pexidartinib) at frequencies of 16 kHz, 22 kHz, 32 kHz, and 40 kHz, respectively, in accordance with the present disclosure.

FIG. 9, panels A-D, staining using Myosin 7a turquoise dye of outer hair cells (OHC, top) and inner hair cells (IHC, bottom) without pexidartinib (left column) and with pexidartinib (right column) in the 16 kHz region, 22.4 kHz region, 32 kHz region, and 40 kHz region, respectively, demonstrating cisplatin mice co-treated with PLX3397 showed greater OHC survival, and the role of macrophages in causing OHC death; panel E shows a graph demonstrating the same, in accordance with the present disclosure.

FIG. 10, panels A-C, show graphs of mass spectrometry measurement of platinum content in microdissected inner ear tissues—spiral ganglion neurons, organ of Corti, and stria vascularis, respectively, demonstrating macrophage ablation by pexidartinib treatment reduces cisplatin entry into the inner ear, in accordance with the present disclosure.

FIG. 11, panel A, shows a schematic depiction of a clinically relevant mouse model corresponding to FIG. 1, panel A; panel B shows images of outer hair cells (top of each image) and inner cell hairs (bottom of each image) at the apex, middle and base of the cochlea for control (top) or cisplatin (bottom), corresponding to FIG. 1, panel D, in accordance with the present disclosure. FIG. 11, panel C, shows a graph of auditory brainstem response (ABR), corresponding to FIG. 1, panel B, in accordance with the present disclosure. FIG. 11, panel D, shows a graph of Distortion OtoAcoustic Emissions (DPOAE), corresponding to FIG. 1, panel C, in accordance with the present disclosure.

FIG. 12, panel A shows an image of a whole cochlear section with panels B and C showing enlarged images for control and cisplatin demonstrating that cisplatin leads to a reduced number of macrophages in the cochlea; panel D shows a bar graph demonstrating the same; corresponding to images in FIG. 6, in accordance with the present disclosure.

FIG. 13 shows images analogous to those of FIG. 9, panels A-D, but with two additional columns for experiments with cisplatin administration, demonstrating cisplatin mice co-treated with PLX3397 showed greater OHC survival, and the role of macrophages in causing OHC death, in accordance with the present disclosure.

FIG. 14, panels A-E, show graphs of mass spectrometry measurement of platinum content in microdissected inner ear tissues—spiral ganglion neurons (SGNs), organ of Corti, stria vascularis, utricle, and saccule, respectively, similar to the graphs shown in FIG. 10, panels A-C, demonstrating macrophage ablation by pexidartinib treatment reduces cisplatin entry into the inner ear, in accordance with the present disclosure.

FIG. 15, panels A-F, showing staining of the cochlea with DAPI, CXCR-GFP, and Kir4.1 (a potassium channel expressed specifically in glial cells) directed stains of saline/vehicle, saline/pexidartinib, cisplatin/vehicle, and cisplatin/pexidartinib in accordance with the present disclosure.

FIG. 16, panels A-H are enlarged images corresponding to the image in panel I of the cochlea with DAPI, CXCR-GFP, and Kir4.1 directed stains of saline/vehicle, saline/pexidartinib, cisplatin/vehicle, and cisplatin/pexidartinib demonstrating the ability of pexidartinib to ablate macrophages, panel J showing a bar graph demonstrating the same in accordance with the present disclosure.

FIG. 17, panels A-F, show saline control and cisplatin experimental images of whole cochlear and cochlear modiolus sections stained with DAPI, CXCR-GFP, and Kir4.1 (a potassium channel expressed specifically in glial cells) directed stains, demonstrating that pexidartinib treatment effectively ablates cochlear macrophages of both saline and cisplatin-treated mice; panel G shows a bar graph demonstrating the same; in accordance with the present disclosure.

FIG. 18 shows another image of myosin7a turquoise staining at the apex, middle and base of the cochlea for control (top) or cisplatin (bottom), corresponding to FIG. 1, panel D, and FIG. 11, panel B, in accordance with the present disclosure.

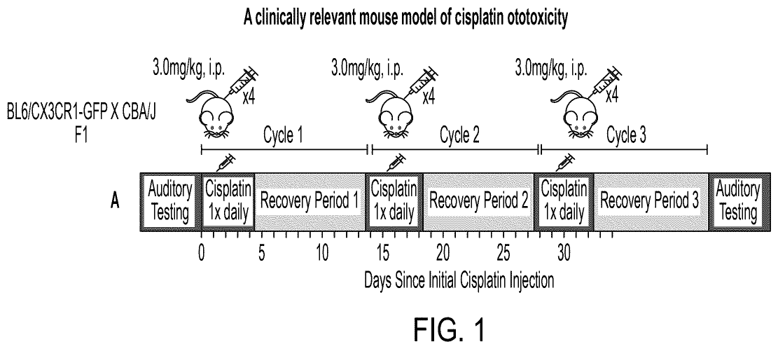

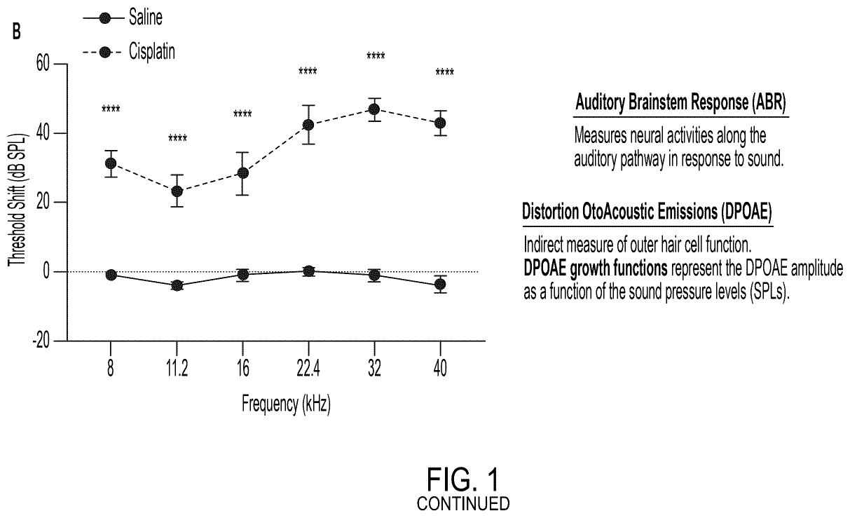

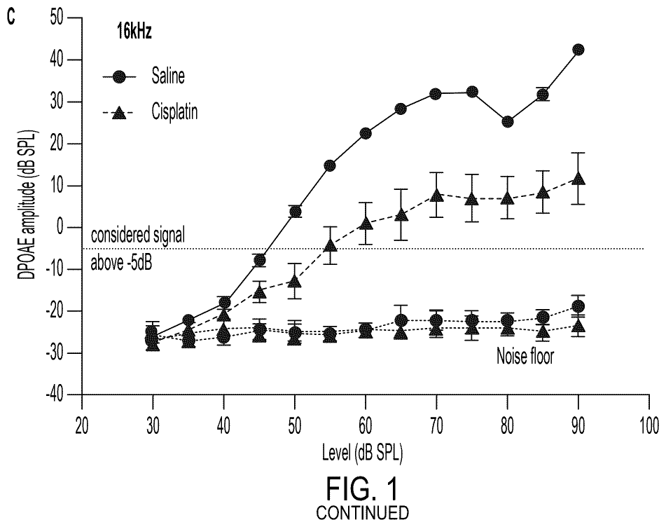

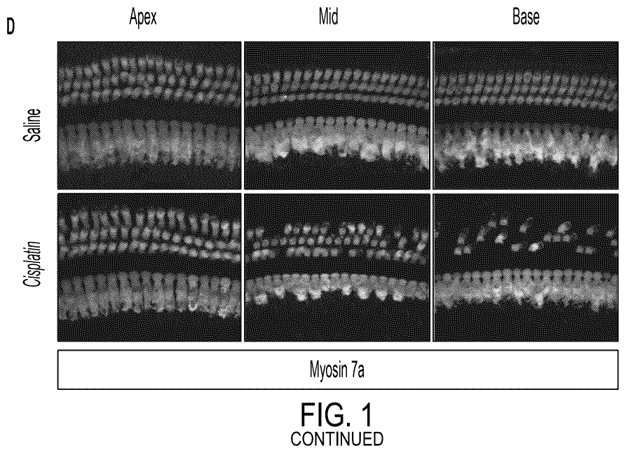

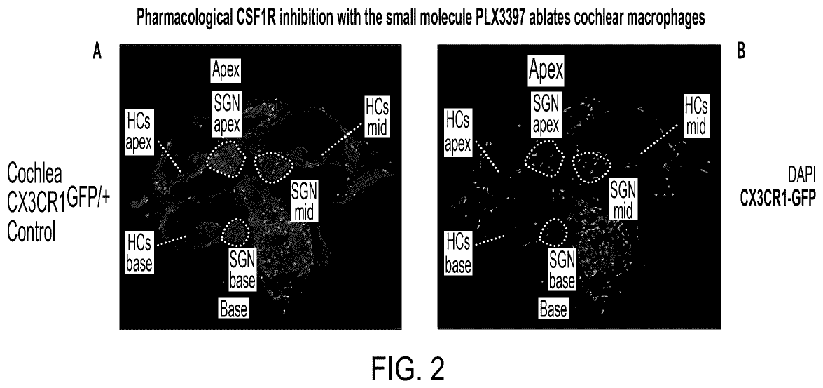

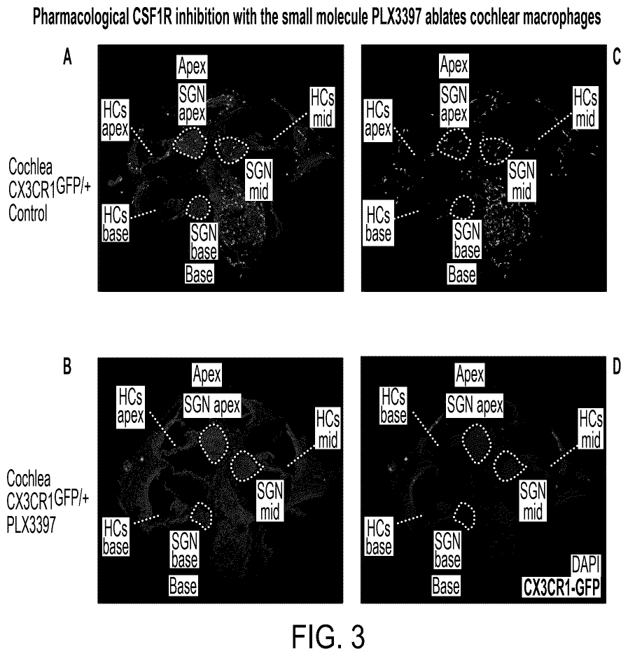

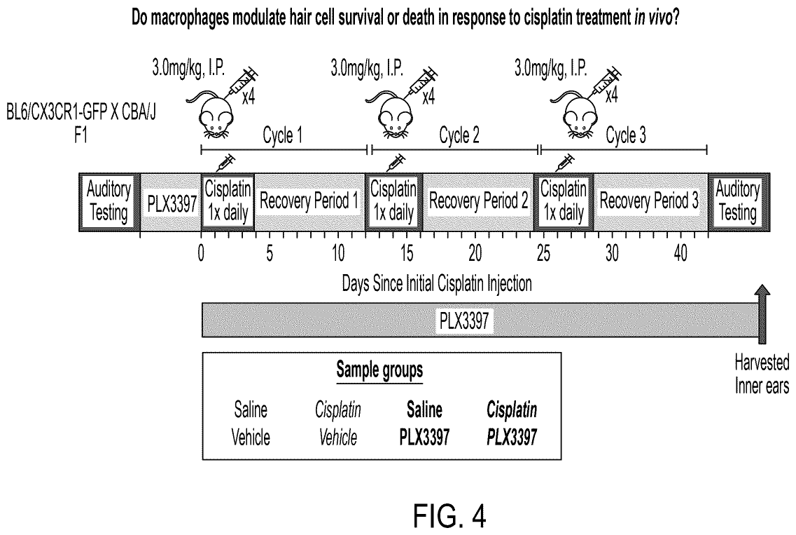

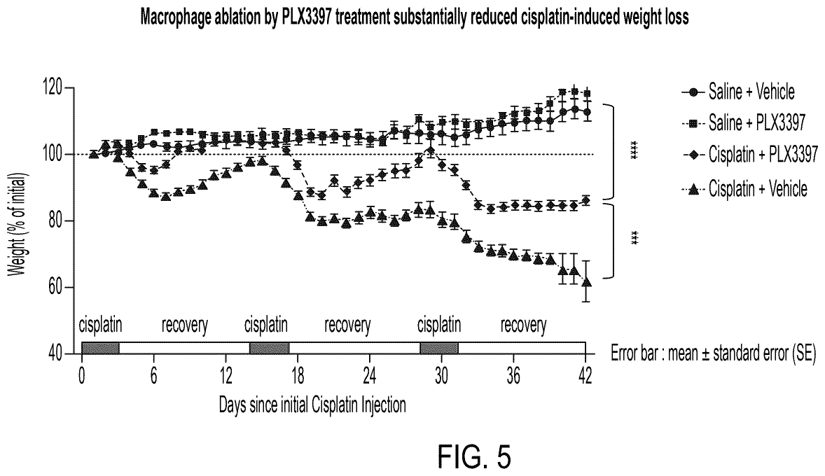

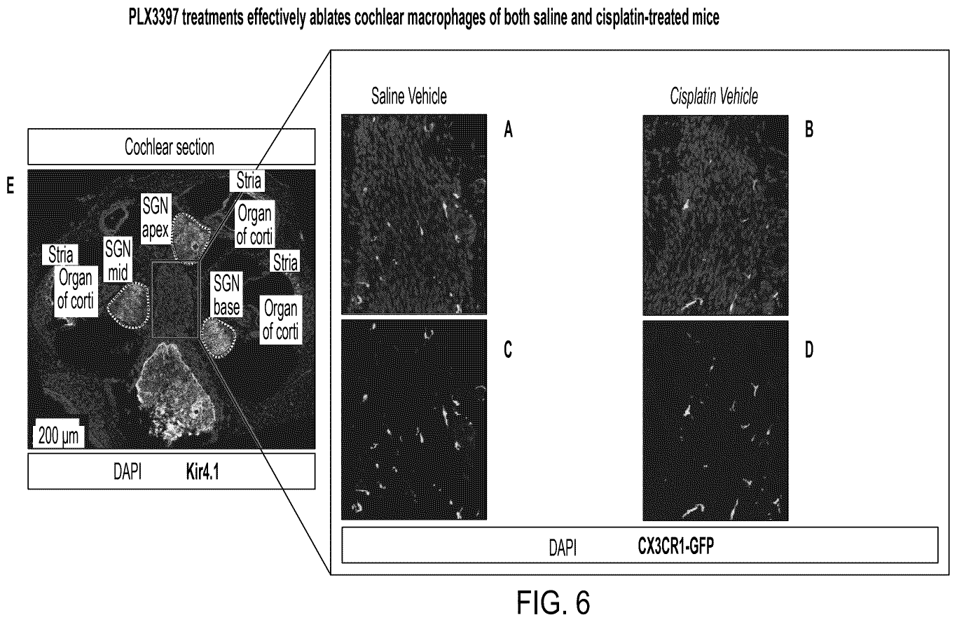

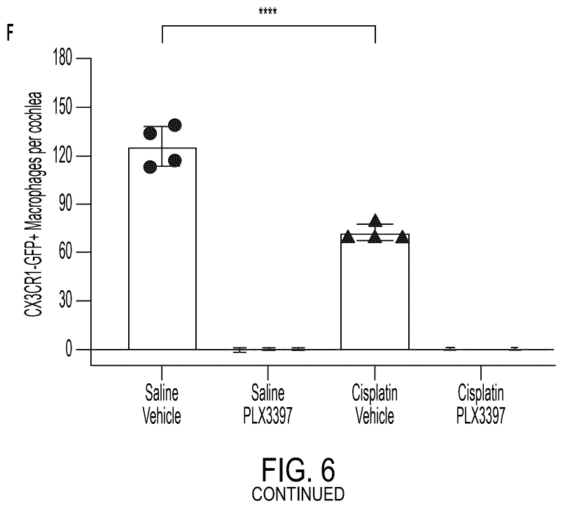

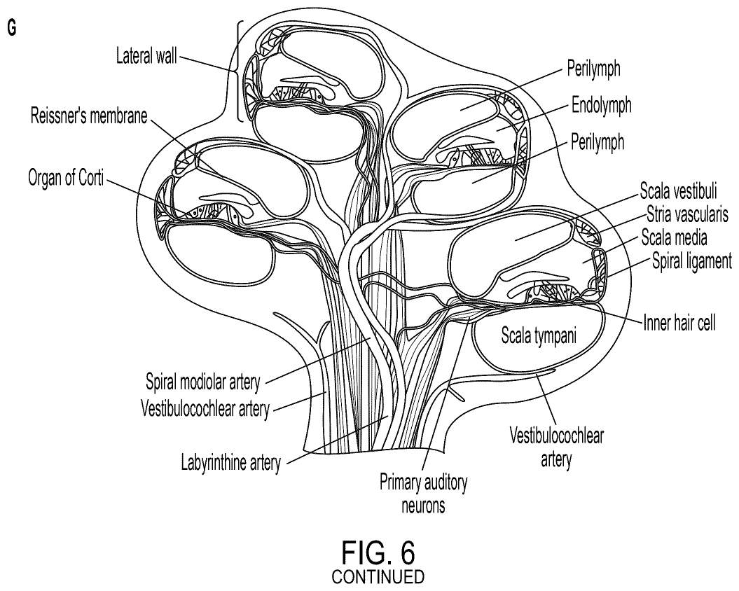

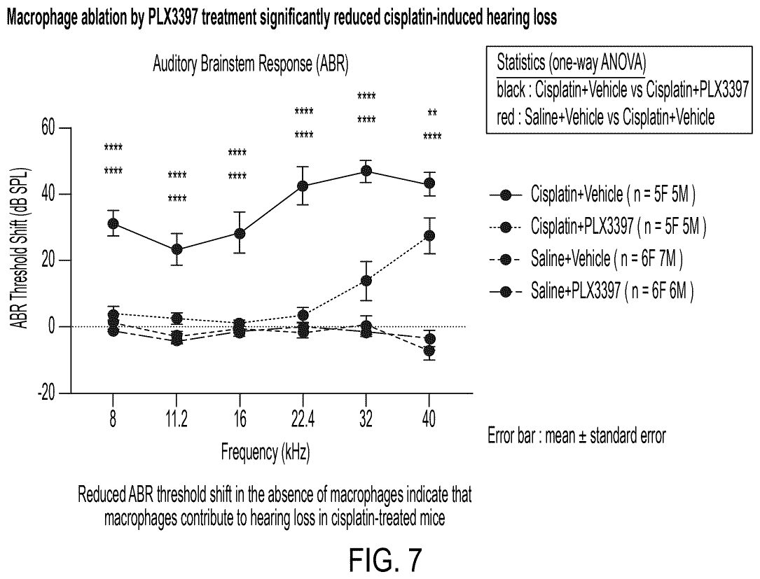

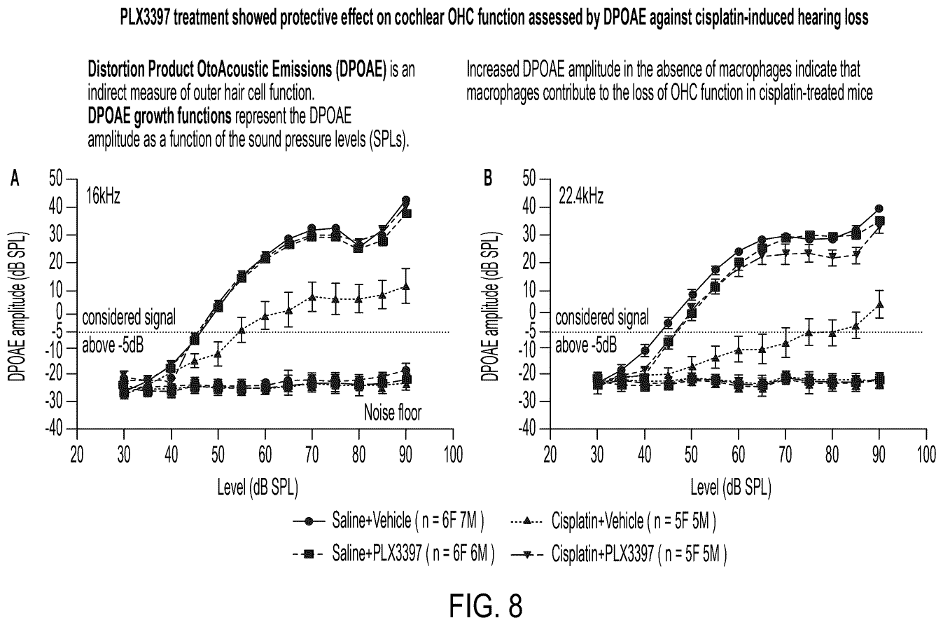

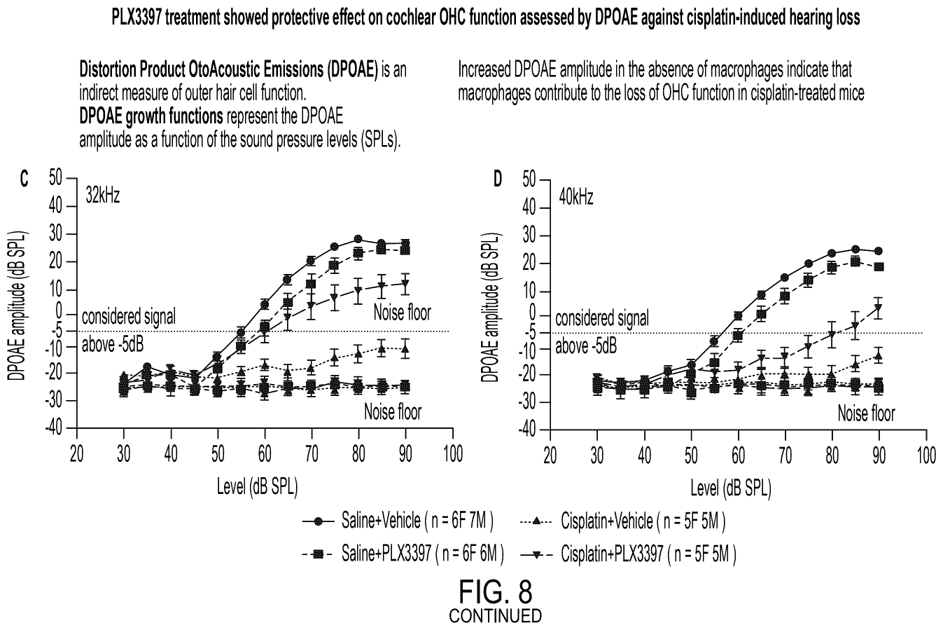

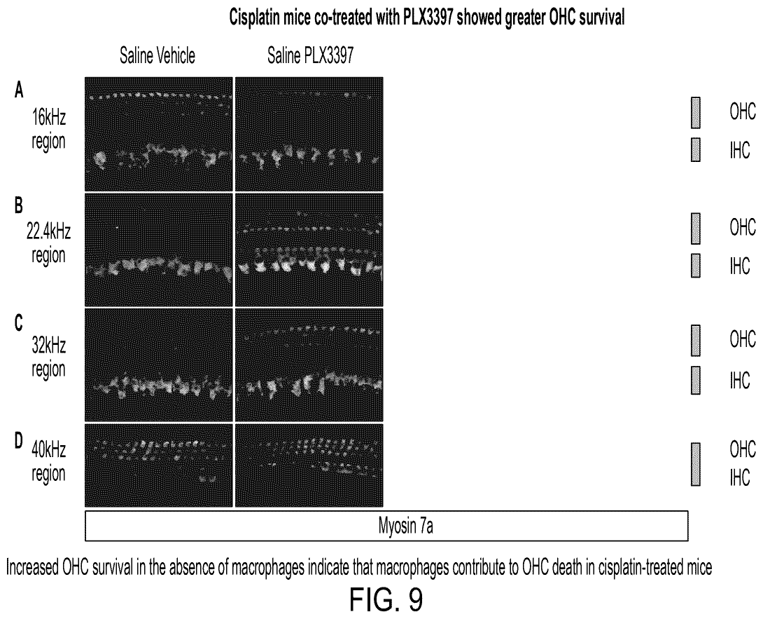

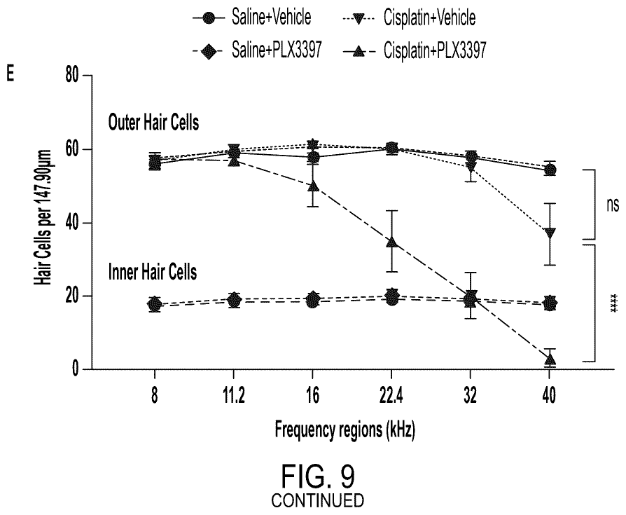

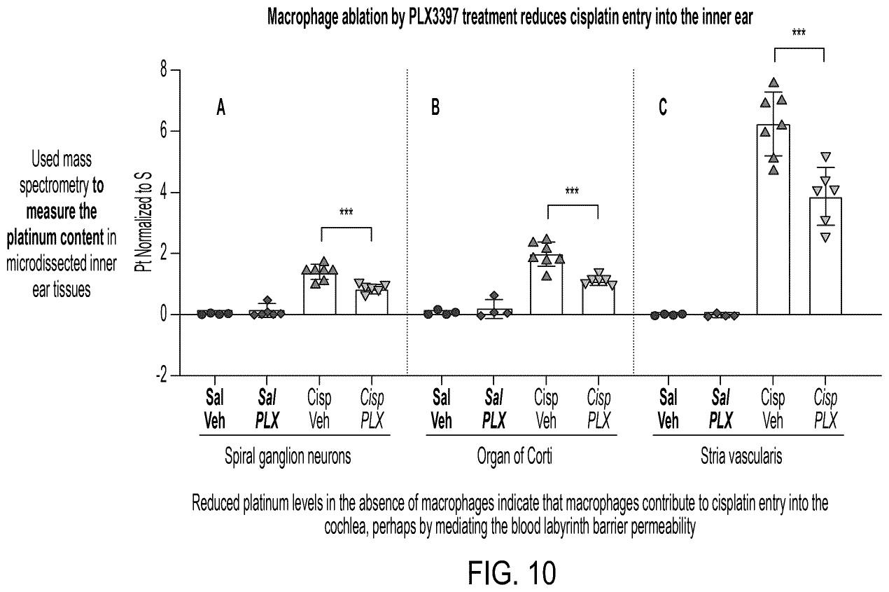

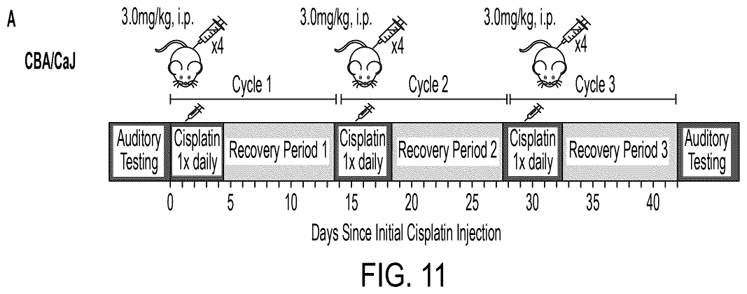

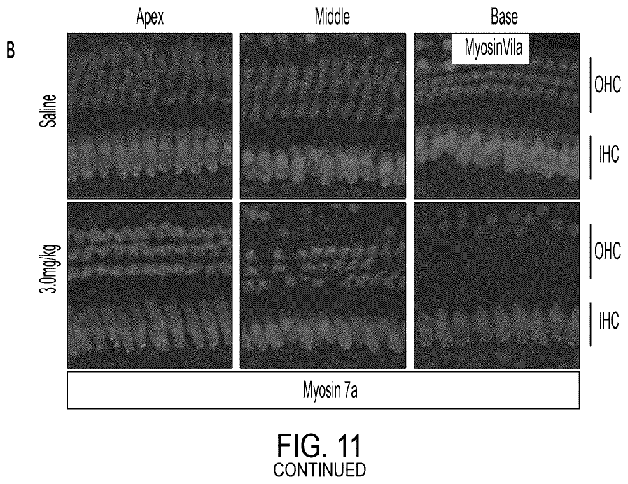

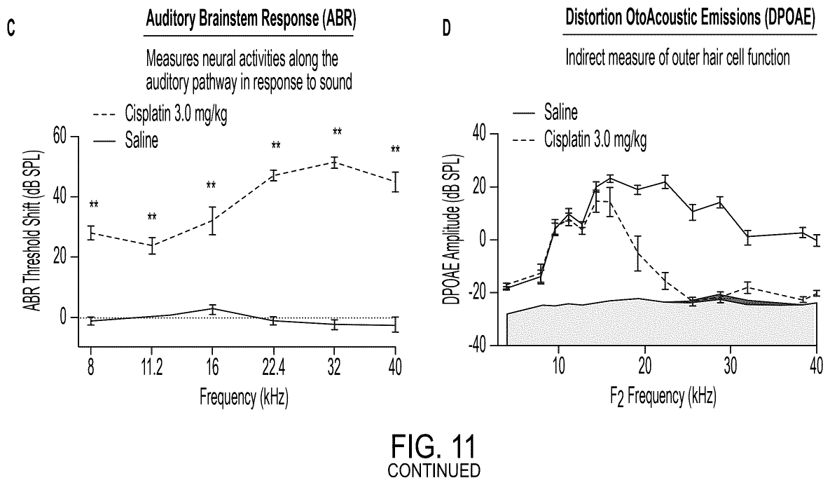

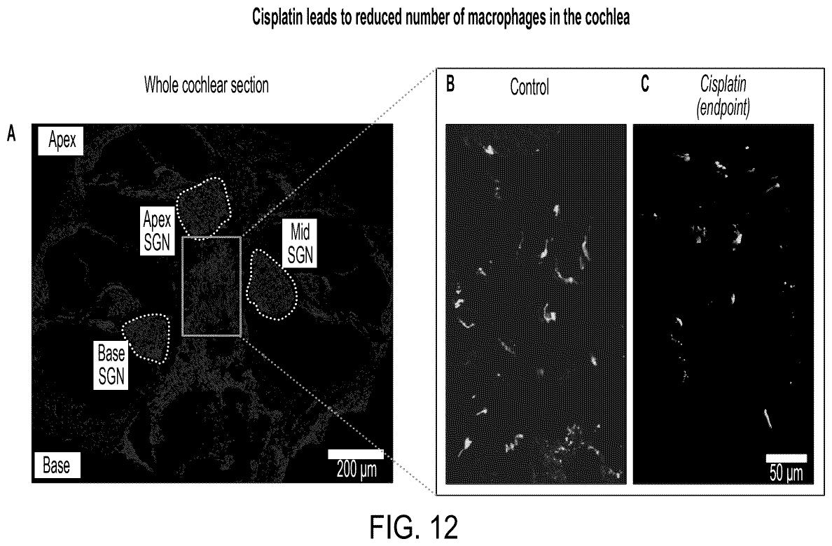

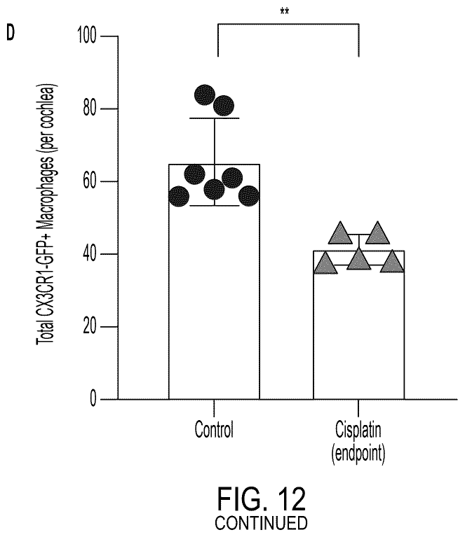

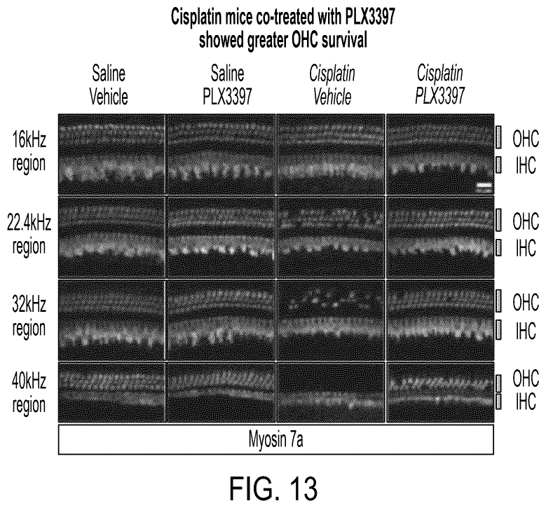

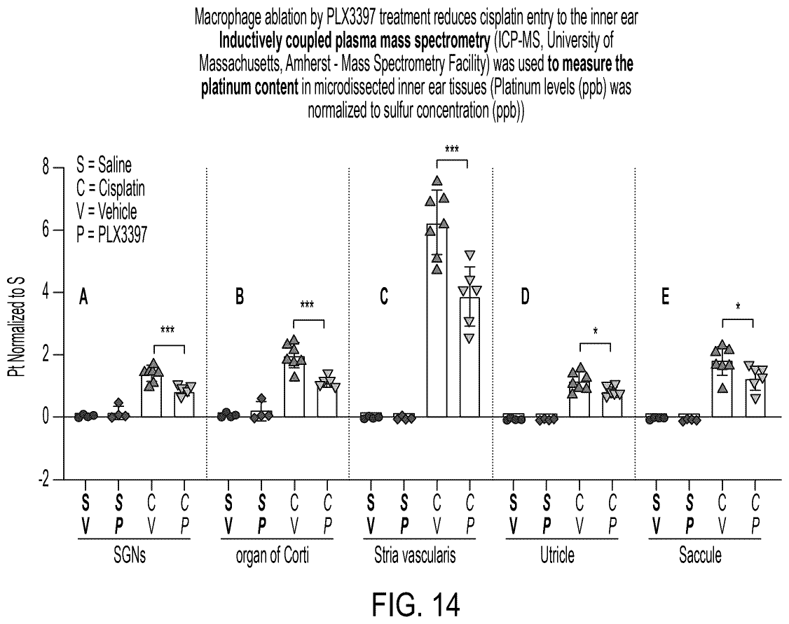

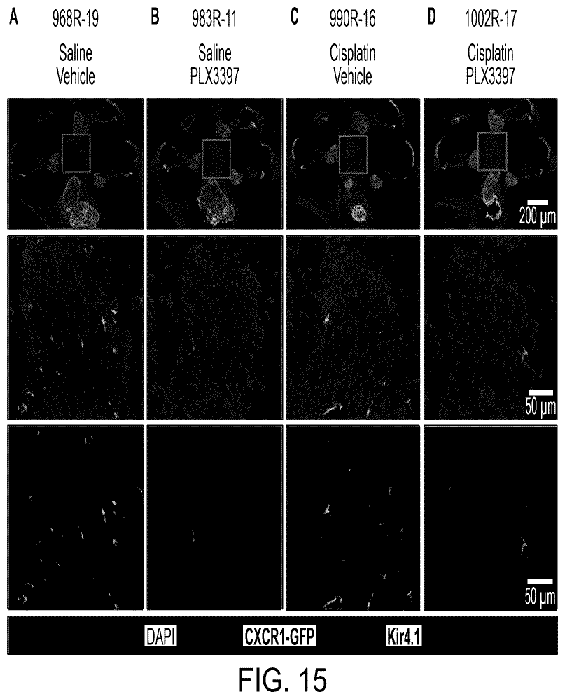

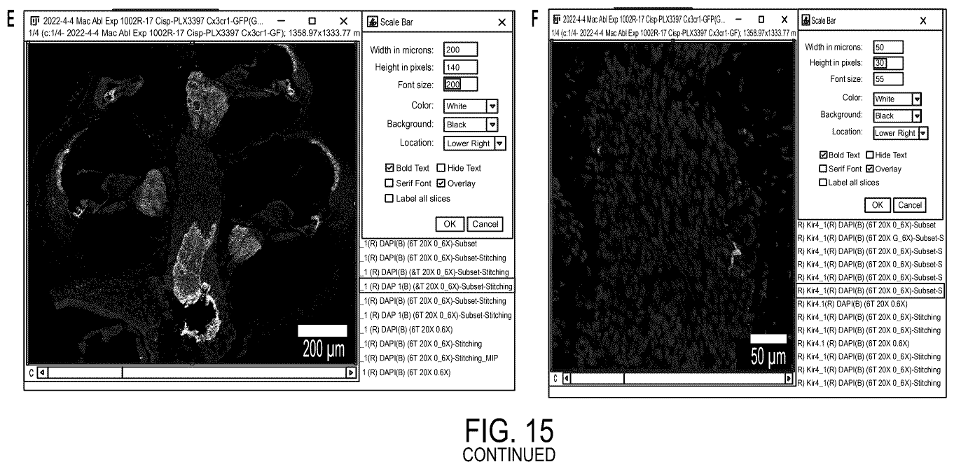

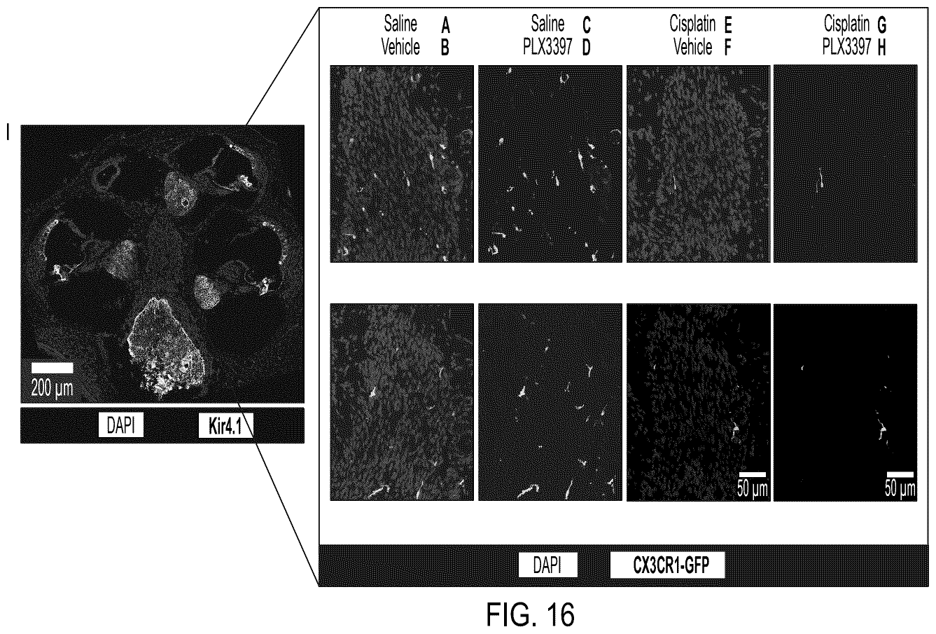

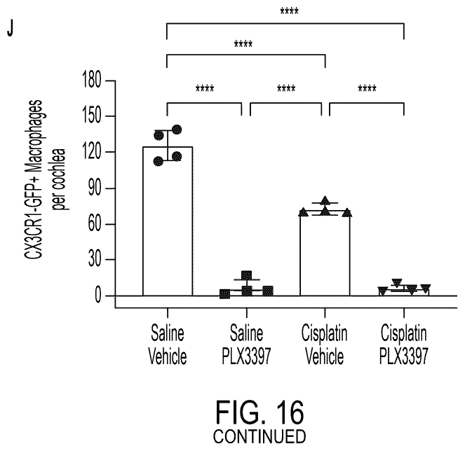

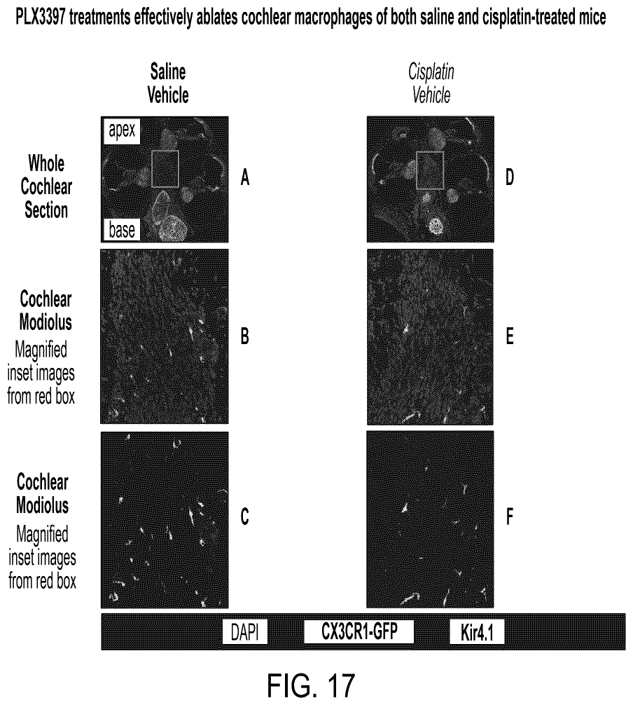

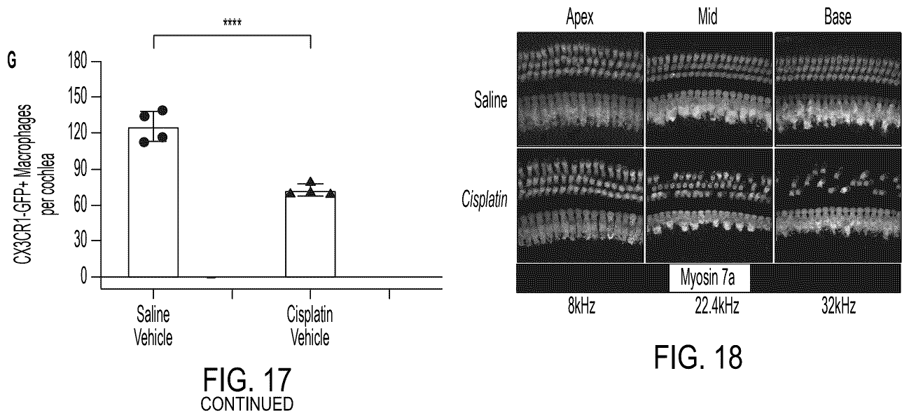

### DETAILED DESCRIPTION

Compounds are described using standard nomenclature. Unless defined otherwise, all technical and scientific terms used herein have the same meaning as is commonly understood by one of skill in the art to which this disclosure belongs.

The terms “a” and “an” do not denote a limitation of quantity, but rather denote the presence of at least one of the referenced items. The term “or” means “and/or”. The terms “comprising,” “having,” “including,” and “containing” are to be construed as open- ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (for example, “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, for example, in Markush group format, each subgroup of the elements is also disclosed, and any element(s) can be removed from the group.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions can be carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids, for example, hydrochloric, hydrobromic, sulfuric, sulfamic, phosphoric, nitric and the like; and the salts prepared from organic acids, for example, acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC-(CH₂) n-COOH where n is 0-4, and the like. Any suitable pharmaceutical salt can be used. The pharmaceutically-acceptable salts of the present compounds can be implemented in any of the preceding or following embodiments, features, or aspects.

As used herein, “prodrug” is intended to include any covalently bonded carriers which release the active parent drug, for example, as according to a compound described herein, or other compounds employed in the methods of the present disclosure in vivo when such prodrug is administered to a mammalian subject. Because prodrugs are known to enhance numerous desirable qualities of pharmaceuticals (for example, solubility, bioavailability, manufacturing, etc.) the compounds employed in the present methods can, if desired, be delivered in prodrug form. Thus, the present disclosure contemplates methods of delivering prodrugs. Prodrugs of the compounds employed in the present disclosure can be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, iso-butyl, sec-butyl, tert-butyl, cyclopropyl, phenyl, benzyl, and phenethyl esters, and the like. The prodrugs of the present compounds can be implemented in any of the preceding or following embodiments, features, or aspects.

Compounds disclosed herein can be administered as the neat chemical, or can be administered as a pharmaceutical composition. Accordingly, the disclosure encompasses pharmaceutical compositions comprising a therapeutically effective amount of a compound or pharmaceutically acceptable salt of a compound, together with at least one pharmaceutically acceptable carrier. The pharmaceutical compositions of the present compounds can be implemented in any of the preceding or following embodiments, features, or aspects. The pharmaceutical composition can contain a therapeutically effective amount of the compound or salt as the only active agent, and can contain at least one additional active agent. The pharmaceutical composition can be in a dosage form that contains from about 0.1 mg to about 2000 mg, from about 10 mg to about 1000 mg, from about 100 mg to about 800 mg, or from about 200 mg to about 600 mg of a compound, and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition can be in a dosage form that contains about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of a compound, and optionally about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of an additional active agent in a unit dosage form. The pharmaceutical composition can also include a molar ratio of a compound and an additional active agent. For example, the pharmaceutical composition can contain a molar ratio of about 0.5:1, about 1:1, about 2:1, about 3:1 or from about 1.5:1 to about 4:1 of an additional active agent to a compound. The dosage forms and molar ratios can be implemented in any of the preceding or following embodiments, features, or aspects.

Compounds disclosed herein can be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition can be formulated as any pharmaceutically useful form, for example, as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, for example, a therapeutically effective amount to achieve the desired purpose. The routes of administration and formulations can be implemented in any of the preceding or following embodiments, features, or aspects.

Carriers include excipients and diluents of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers can be listed in more than one class, for example vegetable oil can be used as a lubricant in some formulations and a diluent in others. Exemplary pharmaceutically acceptable carriers include sugars, starches, celluloses, powdered tragacanth, malt, gelatin, talc, and vegetable oils. Optional active agents can be included in a pharmaceutical composition, which do not substantially interfere with the activity of the compound of the present disclosure. The carriers can be implemented in any of the preceding or following embodiments, features, or aspects.

The pharmaceutical compositions/combinations can be formulated for oral administration. These compositions contain between 0.1 and 99 weight % (wt%) of a compound and usually at least about 5 wt % of a compound, for example, the CSF1R inhibitor and/or platinum-based chemotherapeutic. Compositions can contain from about 25 wt % to about 50 wt % or from about 5 wt % to about 75 wt % of the compound. Compositions can contain about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, or 95 wt % of the compound. The wt % of a compound, for example, the CSF1R inhibitor and/or platinum-based chemotherapeutic, can be implemented in any of the preceding or following embodiments, features, or aspects.

Colony stimulating factor 1 receptor (CSF1R), also known as macrophage colony-stimulating factor receptor (M-CSFR or CD115) is a type Ill protein tyrosine kinase receptor. CSF1R is principally found in cells of the mononuclear phagocyte lineage and macrophages. CSF1R has at least two endogenous ligands, macrophage colony-stimulating factor (M-CSF or CSF1, a cytokine) and IL-34 (also a cytokine and more specifically an interleukin). CSF1R signaling controls differentiation of myeloid cells toward an M2 macrophage phenotype of macrophage. Macrophages, the major resident immune cells in the cochlea that become activated in response to tissue injury, are drivers of both inflammatory and tissue repair responses. CSF1 and signaling through CSF1R can promote proliferation, differentiation, and survival of microglia and macrophages.

Disclosed is a method of impeding platinum-based chemotherapeutic induced ototoxicity, and/or other toxicity. The method can comprise administering a colony stimulating factor 1 receptor (CSF1R) inhibitor to a subject in an amount sufficient to impede ototoxicity, and/or other toxicity, inducible by a platinum-based chemotherapeutic; and administering the platinum-based chemotherapeutic to the subject. Any suitable combination of CSF1R inhibitor and platinum-based chemotherapeutic can be used separately or in admixture. For example, the CSF1R inhibitor can comprise pexidartinib, a prodrug thereof, or a salt thereof, or any combination thereof; the platinum-based chemotherapeutic can comprise cisplatin; and the ototoxicity can comprise hearing loss. The method can be implemented in any of the preceding or following embodiments, features, or aspects.

The method can further comprise diagnosing the subject with a cancer. The subject can have been diagnosed with a cancer. For example, the cancer can comprise a testicular cancer, a bladder cancer, a lung cancer, a stomach cancer, a head & neck cancer, or an ovarian cancer, or any combination thereof. The cancer can comprise a testicular cancer, an ovarian cancer, or a bladder cancer, or any combination thereof. For example, the cancer can comprise a carcinoma, a sarcoma, a myeloma, a leukemia, or a lymphoma, or any combination thereof. The cancer can comprise a solid tumor, a liquid tumor, or both. The cancer can be metastatic. The method can exclude a tenosynovial giant cell tumor (TGCT) or a patient diagnosed with a TGCT. The method can be implemented in any of the preceding or following embodiments, features, or aspects.

Any suitable CSF1R inhibitor can be used. For example, the CSF1R inhibitor can comprise a small molecule therapeutic, a biologic, a prodrug thereof, or a salt thereof, or any combination thereof. The CSF1R inhibitor can comprise a tyrosine kinase inhibitor. Any suitable small molecule can be used. The CSF1R inhibitor can comprise, for example, pexidartinib, edicotinib, PLX647, sotuletinib, vimseltinib, or imatinib, a prodrug thereof, or a salt thereof, or any combination thereof. The CSF-1R inhibitor can comprise pexidartinib, a prodrug thereof, or a salt thereof, or any combination thereof. The CSF1R inhibitor can be implemented in any of the preceding or following embodiments, features, or aspects. The salt can be a pharmaceutically acceptable salt. For example, the pharmaceutically acceptable salt can comprise a hydrochloride salt such as a monohydrochloride salt, or a dihydrochloride salt, or both. The CSF1R inhibitor can comprise any suitable biologic, for example, an antibody or antigen-binding fragment thereof. Any suitable antibody or antigen-binding fragment thereof can be used. For example, the CSF1R inhibitor can comprise a monoclonal antibody, a chimeric antibody, a humanized antibody, or a bifunctional antibody, or any combination thereof. The CSF1R inhibitor can comprise, for example, cabiralizumab, LY3022855, emactuzumab, axatilimab, AMG820, a prodrug thereof, or a salt thereof, or any combination thereof. AMG820 can be excluded as it can cause deafness or otherwise dosed to prevent or minimize the same. The CSF1R inhibitor can further inhibit proto-oncogene receptor kinase (c-KIT), or FMS-like tyrosine kinase 3 with internal tandem duplication mutation (FLT3-ITD), or both. A c-Kit inhibitor, a FLT3-ITD inhibitor, or both can be used in addition to or in the alternative to the CSF1R inhibitor. The CSF1R inhibitor can comprise an inhibitor selective to CSF1R relative to proto-oncogene receptor kinase (c-KIT), or FMS-like tyrosine kinase 3 with internal tandem duplication mutation (FLT3-ITD), or both. The CSF1R inhibitor can comprise a reversible inhibitor, or an irreversible inhibitor, or both. The CSF1R inhibitor can comprise a competitive inhibitor, or a non-competitive inhibitor, or both. The CSF1R inhibitor can antagonize binding to CSF1R, or signaling through CSF1R, or both of CSF1, or IL-34, or both.

Any suitable platinum-based chemotherapeutic or non-platinum-based chemotherapeutic can be used. For example, the platinum-based chemotherapeutic can comprise a chemotherapeutic capable of forming a covalent adduct with DNA of the subject. The platinum-based chemotherapeutic can comprise cisplatin, carboplatin, oxaliplatin, paraplatin, nedaplatin, triplatin tetranitrate, phenanthriplatin, picoplatin, satraplatin, a prodrug thereof, or a salt thereof, or any combination thereof. The platinum-based chemotherapeutic can comprise cisplatin, carboplatin, oxaliplatin, or a prodrug thereof, or a salt thereof, or any combination thereof. The platinum-based chemotherapeutic can comprise cisplatin. The platinum-based chemotherapeutic can be implemented in any of the preceding or following embodiments, features, or aspects.

The ototoxicity induced by the platinum-based ototoxicity can comprise, for example, hearing loss, loss of balance, or tinnitus, or any combination thereof. The hearing loss can comprise sensorineural hearing loss, bilateral hearing loss, progressive hearing loss, irreversible hearing loss, or high frequency hearing loss, or any combination thereof. The ototoxicity can comprise mechanosensory inner ear hair cell toxicity. The mechanosensory inner ear hair cell can comprise an inner hair cell, or an outer hair cell, or both with respect to the cochlear modiolus. The mechanosensory inner ear hair cell can comprise an outer hair cell. The hearing loss can be associated with and/or measured by a threshold shift in auditory brainstem response (ABR). The hearing loss can be associated with and/or measured by a decrease in amplitude of distortion product otoacoustic emissions (DPOAE).

The CSF1R inhibitor can inhibit, directly and/or indirectly, macrophages, or microglia, or both. Inhibition and/or killing of macrophages and/or microglia can be referred to as “ablation.” The macrophages can comprise cochlear resident macrophages, or recruited macrophages, or both. Fractalkine receptor CX3CR1 is a regulator of macrophage function and can be utilized to label macrophages. The resident macrophages can comprise perivascular resident macrophage-like melanocytes (PVM/Ms). PVM/Ms can regulate the integrity of the blood labyrinth barrier (BLB), a barrier between the vasculature and the inner ear fluids (endolymph or perilymph). The BLB can control exchanges between the blood and fluids from intrastitial space in cochlea. The BLB can maintain the inner ear fluid ionic homeostasis. The BLB can restrict entry of most blood-borne (toxic) substances into inner ear tissues. The BLB can selectively pass ions, fluids, and nutrients into cochlea. The CSF1R inhibitor can affect, directly and/or indirectly, the blood labyrinth barrier (BLB) and one or more of its functions.

The method can further comprise testing the hearing, for example, hearing sensitivity, of the subject. Loss of hearing can be partial or full, for example, across standard frequencies of human (or other species subject) hearing or one or more subsets thereof. The testing can focus on particular sound frequencies, especially high-pitched frequencies. Testing the hearing of the subject can be performed before administering the platinum-based chemotherapeutic, or after administering the platinum-based chemotherapeutic, or both. The testing of the hearing of the subject can comprise testing for threshold shifts in auditory brainstem responses (ABR), or amplitude of distortion product-otoacoustic emissions (DPOAE), or both. ABR measures neural activities along the auditory pathway in response to sound. Threshold shifts between tests can be correlated with a loss of hearing. DPOAE is an indirect measure of outer hair cell function. DPOAE growth functions represent the DPOAE amplitude as a function of the sound pressure levels (SPLs). Higher amplitudes can correlate with better hearing.

The amount of CSF1R inhibitor administered can be sufficient to, in addition to or in the alternative to impeding ototoxicity, impede weight loss inducible by the platinum-based chemotherapeutic, impede inner ear hair loss inducible by the platinum- based chemotherapeutic, impede outer hair dysfunction inducible by the platinum-based chemotherapeutic, impede entry of cisplatin into the inner ear, impede hematological toxicity induced by the platinum-based chemotherapeutic, or any combination thereof. The hematological toxicity can comprise, for example, myelosuppression. Hematologic toxicity can, for example, comprise a decrease in bone marrow and blood cells, infection, bleeding, anemia, myelosuppression (bone marrow suppression). Hematologic toxicity can be a side effect of chemotherapy and/or radiation. Hematologic toxicity can be mild or severe. Myeloablation can be fatal in severe cases.

Any suitable dose, dosage form, route of administration, and/or schedule of administration for the CSF1R inhibitor and platinum-based chemotherapeutic can be employed. The dose, dosage form, route of administration, and/or schedule of administration can be implemented in any of the preceding or following embodiments, features, or aspects. The CSF1R inhibitor can be administered before, during, or after, or any combination thereof, administration of the platinum-based chemotherapeutic. For example, the CSF1R inhibitor can be administered at least one minute, at least 10 minutes, at least 30 minutes, at least one hour, at least three hours, at least six hours, at least 12 hours, at least one day, at least three days, at least one week, at least two weeks, at least one month, or longer, or any period therein, or any range therebetween before the platinum-based chemotherapeutic is administered. The platinum-based chemotherapeutic can be administered any suitable period of time, for example, from one time to ten times, from two times to eight times, from one to three times, three times, from one time to five times, or greater, or any intervening number of times, or any range therebetween subsequent to administration of the CSF1R inhibitor. The platinum-based chemotherapeutic can be administered, for example, at least three times subsequent to administration of the CSF1R inhibitor. The CSF1R inhibitor can be administered before one to ten administrations or more of the platinum-based chemotherapeutic. For example, the CSF1R inhibitor can be administered followed by 1, 2, 3, 4, 5, 6, 7, 8, 9, 10, or more administrations of the platinum-based chemotherapeutic. The administration of CSF1R inhibitor can be repeated any number of times. The administration of CSF1R can be repeated after 1, 2, 3, 4, 5, 6, 7, 8, 9, 10, or more administrations of the platinum-based chemotherapeutic, and can be implemented in any of the preceding or following embodiments, features, or aspects.

The CSF1R inhibitor can be administered at any suitable dose. The CSF1R inhibitor can be administered, for example, at a dose of less than 0.5 mg, from about 0.5 mg to about 500 mg per dose, from about 1.0 mg to about 400 mg per dose, from about 10 mg to about 350 mg per dose, from about 25 mg to about 250 mg per dose, from about 50 mg to about 200 mg per dose, from about 100 mg to about 500 mg per dose, from about 200 mg to about 400 mg per dose, or more than 500 mg per dose, or any intervening dose, or any range therebetween. The CSF1R inhibitor can be administered using a solid dosage form, for example, a tablet and/or capsule. For example, pexidartinib monohydrochloride, is marketed by Daiichi Sankyo as TURA LIO for the treatment of adults with symptomatic tenosynovial giant cell tumor (TGCT) as 200 mg capsules-two or three of which is taken a day for a total of 400 mg or 600 mg per day. Pexidartinib and related compounds, as well as their structures, formulae, and synthesis are described in U.S. Pat. Nos. 7,893,075; 8,404,700; 8,461,169; 8,722,702; 9,169,250; and 9,487,515. Pexidartinib and other CSF1R inhibitors can be administered at such doses, or lower, or higher. CSF1R inhibitors can be administered at lower doses sufficient to impede toxicities induced by the platinum-based chemotherapeutic and minimize liver toxicity induced by the CSF1R inhibitor. The dose, the form, or the dose and form can be implemented in any of the preceding or following embodiments, features, or aspects. The method can further comprise monitoring liver toxicity and optionally adjusting the dose of the CSF1R inhibitor accordingly.

The platinum-based chemotherapeutic can be administered at any suitable dosage. The platinum-based chemotherapeutic can be administered, for example, at a dose of less than 0.5 mg, from about 0.5 mg to about 500 mg per dose, from about 1.0 mg to about 400 mg per dose, from about 10 mg to about 350 mg per dose, from about 25 mg to about 250 mg per dose, from about 50 mg to about 200 mg per dose, from about 100 mg to about 500 mg per dose, from about 200 mg to about 400 mg per dose, or more than 500 mg per dose, or any intervening dose, or any range therebetween. The platinum-based chemotherapeutic can be administered, for example, intravenously.

For advanced testicular cancer, the platinum-based chemotherapeutic can be dosed at 20 mg/m² daily for 5 days per cycle. For advanced ovarian cancer, the platinum-based therapeutic can be dosed at 75 mg/m² to 100 mg/m² per cycle once every 3 to 4 weeks. For advanced bladder cancer, the platinum-based chemotherapeutic can be dosed at 50 mg/m² to 70 mg/m² intravenously per cycle once every 3 to 4 weeks. The dose, the form, or the dose and form can be implemented in any of the preceding or following embodiments, features, or aspects. The method can further comprise monitoring renal toxicity and optionally adjusting the dose of the platinum-based chemotherapeutic accordingly. The method can further comprise administering radiation to the subject, or administering at least one non-platinum-based chemotherapeutic to the subject, or both. The chemotherapeutic can be substituted by or used in combination with a non-chemotherapeutic drug associated with ototoxicity, for example, an antibiotic.

The CSF1R inhibitor and platinum-based chemotherapeutic can be administered separately or in admixture. They can be administered using the same or different dosage forms and/or route of administration. The subject to which the CSF1R inhibitor and platinum-based chemotherapeutic are administered to can be any desired animal. The subject can be a mammal, for example, a rodent (e.g., mouse or rat), a lagomorph (e.g., rabbit), a ruminate, or a primate. The subject can be a companion animal, for example, a bird, a guinea pig, a hamster, a ferret, a cat, a dog, a pig, or a horse. The method subject can be a human. The human subject can be infant, toddler, juvenile, adolescent, adult, or senior. The human subject can be 0 to 6 months old, 6 months old to 2 years old, 1 year old to 5 years old, 1 year to 12 years, 12 years to 18 years, 18 years to 40 years, 25 years to 60 years, 60 years old to 85 years old, 80 years old to 100 years old, or more.

A pharmaceutical composition is provided that can comprise the CSF1R inhibitor, the platinum-based therapeutic, and a pharmaceutically acceptable excipient. A kit is provided that can comprise the CSF1R inhibitor and the platinum-based chemotherapeutic.

A method of screening for a compound able to impede a platinum-based chemotherapeutic induced toxicity is provided. The method can comprise the following steps. A physiologic parameter can be measured in a non-human subject to obtain a first measurement, the physiological parameter associated with the toxicity and deleteriously affected by a platinum-based chemotherapeutic. A test compound can be administered to the non-human subject after measuring the physiologic parameter. The platinum-based chemotherapeutic can be administered to the non-human subject after measuring the physiologic parameter. The physiologic parameter can be measured in the non-human subject subsequent to administration of the test compound and the platinum-based chemotherapeutic to obtain a second measurement. The first and second measurements are compared, a smaller deleterious change, than that experienced by a non-human subject not administered the test compound, indicating the test compound as a therapeutic capable of impeding the toxicity. The toxicity can be, for example, an ototoxicity.

A therapeutic identified by the method of any preceding or following embodiment/feature/aspects is provided. A colony stimulating factor 1 receptor (CSF1R) inhibitor is provided for use in impeding ototoxicity inducible by a platinum-based chemotherapeutic. Use of a colony stimulating factor 1 receptor (CSF1R) inhibitor is provided for impeding ototoxicity inducible by a platinum-based chemotherapeutic. Use of a colony stimulating factor 1 receptor (CSF1R) inhibitor is provided for manufacture of a medicament to impede ototoxicity inducible by a platinum-based chemotherapeutic.

### EXAMPLES

A clinically relevant mouse model of cisplatin-induced ototoxicity was developed and used to examine whether macrophages play a role in hearing loss in response to cisplatin. Pexidartinib is an inhibitor of the colony stimulating factor 1 receptor (CSF1R), which aids in the survival of microglia and cochlear resident macrophages.

Hearing sensitivity was assessed prior to pexidartinib/cisplatin administration and after the end of the third cycle of the treatment protocol using auditory brainstem responses (ABR) and distortion product otoacoustic emissions (DPOAE). Macrophage ablation by pexidartinib treatment significantly reduced hearing loss (ABR) protected from the loss of OHC function (DPOAE) increased OHC survival reduced cisplatin entry to the inner ear Macrophages contributed to hearing loss and OHC death in cisplatin-treated mice. Histology can be performed using MyosinVIla (turquoise).

The data are supportive of macrophage ablation being protective against cisplatin-induced hearing loss. ABR threshold shifts, indicative of hearing loss, were significantly reduced in mice treated with both cisplatin and pexidartinib compared to mice treated with cisplatin only. similarly, mice treated with both cisplatin and pexidartinib showed higher DPOAE amplitudes compared to mice treated with cisplatin only, suggesting that macrophage ablation protected against cisplatin-induced outer hair cell dysfunction. Quantification of cochlear hair cells is supportive of the protective effect of macrophage ablation by pexidartinib treatment due to protection of outer hair cells against cisplatin-induced hair cell loss. Together, these findings demonstrate that macrophages contribute to cisplatin-induced hearing loss. Data and other information relevant to the experimental examples are depicted in the various figures of the drawings and summarized herein.

The present disclosure can include any combination of these various features or embodiments above and/or below as set forth in sentences and/or paragraphs. Any combination of disclosed features herein is considered part of the present disclosure. Further, when an amount, concentration, or other value or parameter is given as either a range, or a list of upper values and lower values, all ranges formed from any pair of any upper range limit or value and any lower range limit or value are also disclosed, regardless of whether ranges are separately disclosed. Where a range of numerical values is recited herein, unless otherwise stated, the range is intended to include the endpoints thereof, and all subranges, integers, and fractions within the range. The scope of the disclosure is not limited to the specific values recited in a range. All references cited in this specification are herein incorporated by reference as though each reference was specifically and individually indicated to be incorporated by reference. Each of the elements described herein, or two or more together, are also within the scope of the present disclosure.

What is claimed is:

1. A method of impeding platinum-based chemotherapeutic induced ototoxicity, the method comprising:
administering a colony stimulating factor 1 receptor (CSF1R) inhibitor to a subject in an amount sufficient to impede ototoxicity inducible by a platinum-based chemotherapeutic; and
administering the platinum-based chemotherapeutic to the subject;
optionally diagnosing the subject with cancer; and
optionally administering radiation to the subject, or administering at least one non-platinum-based chemotherapeutic to the subject, or both;
wherein optionally the CSF1R inhibitor comprises pexidartinib, a prodrug thereof, or a salt thereof, or any combination thereof;
wherein optionally the platinum-based chemotherapeutic comprises cisplatin; and
wherein optionally the ototoxicity comprises hearing loss.

2-3. (canceled)

4. The method of claim 1, wherein optionally the subject has been diagnosed with a cancer-prior to the administering of the colony stimulating factor 1 receptor (CSF1R);
wherein optionally the subject is diagnosed with cancer after the administering of the platinum-based chemotherapeutic;
wherein optionally the cancer comprises testicular cancer, a bladder cancer, a lung cancer, a stomach cancer, a head & neck cancer, an ovarian cancer, a carcinoma, a sarcoma, a myeloma, a leukemia, or a lymphoma, or any combination thereof;
wherein optionally the cancer comprises a solid tumor or a liquid tumor;
wherein optionally the cancer is metastatic;
wherein optionally the cancer excludes a tenosynovial giant cell tumor (TGCT).

5-11. (canceled)

12. The method of claim 1, wherein the CSF1R inhibitor comprises a small molecule therapeutic, a biologic, a prodrug thereof, or a salt thereof, or any combination thereof;
wherein optionally the salt is a pharmaceutically acceptable salt;
wherein optionally the salt comprises a hydrochloride salt;
wherein optionally the salt comprises a monohydrochloride salt, or a dihydrochloride salt, or both;
wherein optionally the salt is a monohydroxychloride salt.

13. (canceled)

14. The method of claim 12, wherein the CSF1R inhibitor comprises a tyrosine kinase inhibitor;
wherein optionally the CSF1R inhibitor comprises pexidartinib, edicotinib PLX647, sotuletinib, vimseltinib, or imatinib, a prodrug thereof, or a salt thereof, or any combination thereof.

15-22. (canceled)

23. The method of claim 12, wherein the CSF1R inhibitor comprises an antibody or antigen-binding fragment thereof;
wherein optionally the CSF1R inhibitor comprises a monoclonal antibody;
wherein optionally the CSF1R inhibitor comprises a chimeric antibody;
wherein optionally the CSF1R inhibitor comprises a humanized antibody;
wherein optionally the CSF1R inhibitor comprises cabiralizumab, LY3022855, emactuzumab, axatilimab, AMG820, a prodrug thereof, or a salt thereof, or any combination thereof.

24-28. (canceled)

29. The method of claim 12, wherein the CSF1R inhibitor comprises an inhibitor selective to CSF1R relative to proto-oncogene receptor kinase (c-KIT), or FMS-like tyrosine kinase 3 with internal tandem duplication mutation (FLT3-ITD), or both.

30. The method of claim 12, wherein the CSF1R inhibitor comprises a reversible inhibitor, or an irreversible inhibitor, or both;
wherein optionally the CSF1R inhibitor comprises a competitive inhibitor, or a non-competitive inhibitor, or both;
wherein optionally the CSF1R inhibitor antagonizes binding to CSF1R, or signaling through CSF1R, or both of CSF1, or IL-34, or both.

31-32. (canceled)

33. The method of claim 1, wherein the platinum-based chemotherapeutic comprises a chemotherapeutic capable of forming a covalent adduct with DNA of the subject;
wherein optionally the platinum-based chemotherapeutic comprises cisplatin, carboplatin, oxaliplatin, paraplatin, nedaplatin, triplatin tetranitrate, phenanthriplatin, picoplatin, satraplatin, a prodrug thereof, or a salt thereof, or any combination thereof;
wherein optionally the platinum-based chemotherapeutic is administered from one time to ten times subsequent to administration of the CSF1R inhibitor;
wherein optionally the platinum-based chemotherapeutic is administered at least three times subsequent to administration of the CSF1R inhibitor.

34-37. (canceled)

38. The method of claim 1, wherein the ototoxicity comprises hearing loss, loss of balance, or tinnitus, or any combination thereof;
wherein optionally the hearing loss comprises sensorineural hearing loss, bilateral hearing loss, progressive hearing loss, irreversible hearing loss, or high frequency hearing loss, or any combination thereof;
wherein optionally the ototoxicity comprises mechanosensory inner ear hair cell toxicity:
wherein optionally the hearing loss is associated with a threshold shift in auditory brainstem response (ABR);
wherein optionally the hearing loss is associated with a decrease in amplitude of distortion product otoacoustic emissions (DPOAE); or
wherein optionally the mechanosensory inner ear hair cell comprises an inner hair cell, or an outer hair cell, or both.

39-45. (canceled)

46. The method of claim 12, wherein the CSF1R inhibitor inhibits macrophages, or microglia, or both;
wherein optionally the macrophages comprise cochlear resident macrophages, or recruited macrophages, or both;
wherein optionally the resident macrophages comprise perivascular resident macrophage-like melanocytes (PVM/Ms);
wherein optionally the CSF1R inhibitor affects the blood labyrinth barrier (BLB);
wherein optionally the CSF1R inhibitor inhibits proto-oncogene receptor kinase (c-KIT), or FMS-like tyrosine kinase 3 with internal tandem duplication mutation (FLT3-ITD), or both; or
wherein optionally a c-KIT inhibitor, a FLT3-ITD inhibitor, or both are administered in addition to the CSF1R inhibitor.

47-50. (canceled)

51. The method of claim 1, further comprising testing the hearing of the subject;
wherein optionally testing the hearing of the subject is performed before administering the platinum-based chemotherapeutic, or after administering the platinum-based chemotherapeutic, or both;
wherein optionally testing the hearing of the subject comprises testing for threshold shifts in auditory brainstem responses (ABR), or amplitude of distortion product-otoacoustic emissions (DPOAE), or both.

52-53. (canceled)

54. The method of claim 1, wherein the amount of CSF1R inhibitor administered is sufficient to impede weight loss inducible by the platinum-based chemotherapeutic, impede inner ear hair loss inducible by the platinum-based chemotherapeutic, impede outer hair dysfunction inducible by the platinum-based chemotherapeutic, impede entry of cisplatin into the inner ear, impede hematological toxicity induced by the platinum-based chemotherapeutic, or any combination thereof;
wherein optionally the hematological toxicity comprises myelosuppression.

55. (canceled)

56. The method of claim 1, wherein the CSF1R inhibitor is administered before, during, or after, or any combination thereof, administration of the platinum-based chemotherapeutic;
wherein optionally the CSF1R inhibitor is administered before one to ten administrations of the platinum-based chemotherapeutic;
wherein optionally the CSF1R inhibitor is administered at from about 0.5 mg to about 500 mg per dose;
wherein optionally the CSF1R inhibitor is administered in solid dosage form; or
wherein optionally the CSF1R inhibitor and platinum-based chemotherapeutic are administered in admixture.

57-62. (canceled)

63. The method of claim 1, wherein the platinum-based chemotherapeutic is administered at from about 0.5 mg to about 500 mg per dose;
wherein optionally the platinum-based chemotherapeutic is administered intravenously;
wherein optionally the CSF1R inhibitor and platinum-based chemotherapeutic are administered in admixture.

64-65. (canceled)

66. The method of claim 1, wherein the subject is a mammal;
wherein optionally the subject is a rodent or a primate;
wherein optionally the subject is a companion animal;
wherein optionally the subject is a human;
wherein optionally the subject is a juvenile;
wherein optionally the subject is an adult.

67-72. (canceled)

73. A pharmaceutical composition comprising the CSF1R inhibitor, the platinum-based therapeutic, and a pharmaceutically acceptable excipient.

74. A kit comprising the CSF1R inhibitor and the platinum-based chemotherapeutic.

75. A method of screening for a compound able to impede a platinum-based chemotherapeutic induced toxicity, the method comprising:
measuring a physiologic parameter in a non-human subject to obtain a first measurement, the physiological parameter associated with the toxicity and deleteriously affected by a platinum-based chemotherapeutic;
administering a test compound to the non-human subject after measuring the physiologic parameter;
administering the platinum-based chemotherapeutic to the non-human subject after measuring the physiologic parameter;
measuring the physiologic parameter in the non-human subject subsequent to administration of the test compound and the platinum-based chemotherapeutic to obtain a second measurement; and
comparing the first and second measurements, a smaller deleterious change, than that experienced by a non-human subject not administered the test compound, indicating the test compound as a therapeutic capable of impeding the toxicity;
wherein optionally the toxicity is an ototoxicity.

76-77. (canceled)

78. A colony stimulating factor 1 receptor (CSF1R) inhibitor for use in impeding ototoxicity inducible by a platinum-based chemotherapeutic.

79. (canceled)

80. Use of a colony stimulating factor 1 receptor (CSF1R) inhibitor A process for manufacture of a medicament comprising a colony stimulating factor 1 receptor (CSF1R) inhibitor to impede ototoxicity inducible by a platinum-based chemotherapeutic.
